SEQUENCE LISTING (6 sequences)

SEQ ID NO: 1 - protein, 359 aa, synthetic construct
MHLDMPEYWDRETQNYKRQAQADRVSLRNLRGYYNQSEDGSHTLQRMYGCDLGPDGRLLRGYDQSAYDGNDYIALNEVLRSWTAADTAAQITQRKLEAARAAEIGSDLDRSENIMPVLVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 2 - protein, 10 aa, synthetic construct
DYIALNEDLR

SEQ ID NO: 3 - protein, 10 aa, synthetic construct
DYIALNEVLR

SEQ ID NO: 4 - protein, 9 aa, synthetic construct
DYIALENLR

SEQ ID NO: 5 - protein, 18 aa, synthetic construct
AYDGNDYIALNEVLRSWT

SEQ ID NO: 6 - protein, 106 aa, synthetic construct
MPEYWDRETQNYKRQAQADRVSLRNLRGYYNQSEDGSHTLQRMYGCDLGPDGRLLRGYDQSAYDGNDYIALNEVLRSWTAADTAAQITQRKLEAARAAEIGSDLDR

(19) United States
(12) Patent Application Publication
(10) Pub. No.: US 2024/0199720 A1
(43) Pub. Date: Jun. 20, 2024

(54) COMPOSITION AND METHOD FOR PREVENTING OR TREATING SARS-COV-2 INFECTION

(71) Applicant: National Yang Ming Chiao Tung University, Hsinchu (TW)

(72) Inventors: Chia-Ching CHANG, Hsinchu (TW); Chia-Yu CHANG, Hsinchu (TW); Shin-Ru SHIH, Hsinchu (TW); Sheng-Yu HUANG, Hsinchu (TW)

(21) Appl. No.: 18/540,384

(22) Filed: Dec. 14, 2023

Related U.S. Application Data
(60) Provisional application No. 63/387,452, filed on Dec. 14, 2022.

(51) Int. Cl. C07K 14/74

(52) U.S. Cl. CPC C07K 14/70539; A61K 38/00

(57) ABSTRACT

Provided is a non-natural polypeptide including a fragment of major histocompatibility complex class I (MHC I). Also provided are a method for inhibiting combination between SARS-COV-2 and a cell of a subject and a method for preventing or treating SARS-COV-2 infection in a subject in need thereof, including contacting the cell or administering to the subject with the non-natural polypeptide.

### CROSS-REFERENCE TO RELATED APPLICATION

This application claims priority to U.S. provisional patent application No. 63/387,452, filed on Dec. 14, 2022, the entire content of which is incorporated herein by reference.

### REFERENCE TO AN ELECTRONIC SEQUENCE LISTING

The content of the electronic sequence listing (sequence listing.xml; Size: 6,790 bytes; and Date of Creation: Dec. 14, 2023) is herein incorporated by reference in its entirety.

### BACKGROUND

### Technical Field

The present disclosure relates to the fields of molecular biology, protein chemistry, immunochemistry, and pharmacology and involves methods and compositions for prevention and treatment of coronavirus disease 2019 (COVID-19), a disease caused by infection of severe acute respiratory syndrome coronavirus 2 (SARS-COV-2).

### Description of Related Art

Coronavirus disease 2019 (COVID-19) is an infectious disease caused by severe acute respiratory syndrome coronavirus 2 (SARS-COV-2). Since first identified in December 2019 in Wuhan, Hubei, China, the disease has caused a worldwide pandemic. The World Health Organization (WHO) declared the COVID-19 outbreak a public health emergency of international concern (PHEIC) on Jan. 30, 2020 and a pandemic on Mar. 11, 2020. As of September 2022, more than 600 million cases have been reported across 188 countries and territories with more than 3 million deaths.

Because there are no effective treatments for COVID-19 yet, an effective treatment or preventive measure for COVID-19 remains a common goal of the scientists and researchers worldwide.

For an efficient containment of a pandemic disease, it is necessary to prepare an effective medication that can prevent and treat SARS-COV-2 and their variants.

### SUMMARY

The present disclosure relates to a use of MHC class I (MHC I) polypeptides and fragments thereof as an inhibitor for inhibiting the combination of SARS-COV-2 and a cell in a subject. The present disclosure also relates to a use of MHC class I (MHC I) polypeptides and fragments thereof as compositions for preventing or treating COVID-19. The present disclosure further relates to prevention or treatment of SARS-COV-2 infection using MHC class I (MHC I) polypeptides and fragments thereof. The present disclosure still further relates to a method of preventing or treating SARS-COV-2 infection by inhibiting binding of SARS-COV-2 to HLA-C on a cell surface.

Accordingly, a non-natural polypeptide for inhibiting the combination of SARS-COV-2 and a cell in a subject, and methods for treating COVID-19 and preventing infection caused by SARS-COV-2 and thereby preventing COVID-19 are provided.

In accordance with the non-natural polypeptide of the present disclosure, a fragment of MHC class I (MHC I) is included.

In at least one embodiment of the present disclosure, a non-natural polypeptide for inhibiting the combination of SARS-COV-2 and a cell in a subject is provided, wherein the non-natural polypeptide includes a fragment of MHC class I (MHC I). In at least one embodiment of the present disclosure, the non-natural polypeptide has an amino acid sequence of SEQ ID NO: 1. In at least one embodiment of the present disclosure, the non-natural polypeptide includes an amino acid sequence of SEQ ID NOs: 2, 3, 4, 5, or 6. In at least one embodiment of the present disclosure, the non-natural polypeptide includes an amino acid sequence that has at least 50%, at least 55%, at least 60%, at least 65%, at least 70%, at least 72%, at least 74%, at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 2, 3, 4, 5, or 6. In at least one embodiment of the present disclosure, the non-natural polypeptide has at least 50%, at least 55%, at least 60%, at least 65%, at least 70%, at least 72%, at least 74%, at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to MHC I. In at least one embodiment of the present disclosure, the non-natural polypeptide has at least 50%, at least 55%, at least 60%, at least 65%, at least 70%, at least 72%, at least 74%, at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1.

In at least one embodiment of the present disclosure, the non-natural polypeptide shows binding activity to SARS-COV-2 spike protein receptor binding domain (RBD).

In at least one embodiment of the present disclosure, the non-natural polypeptide has a length of 7 amino acids to 2,000 amino acids, e.g., 8 amino acids, 9 amino acids, 10 amino acids, 15 amino acids, 18 amino acids, 20 amino acids, 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 355 amino acids, 358 amino acids, 359 amino acids, 360 amino acids, 365 amino acids, 375 amino acids, or 400 amino acids. In at least one embodiment of the present disclosure, the non-natural polypeptide consists of 10 to 150 amino acids, 10 to 200 amino acids, 20 to 250 amino acids, 20 to 300 amino acids, 30 to 350 amino acids, 30 to 400 amino acids, 40 to 450 amino acids, or 40 to 500 amino acids. In at least one embodiment of the present disclosure, the non-natural polypeptide consists of 10 to 400 amino acids.

In at least one embodiment of the present disclosure, the non-natural polypeptide inhibits the combination of SARS-COV-2 and a cell membrane receptor of a cell in the subject. In at least one embodiment of the present disclosure, the cell membrane receptor of the cell in the subject is angiotensin-converting enzyme II (ACE2).

In at least one embodiment of the present disclosure, the non-natural polypeptide inhibits the combination of SARS-COV-2 and a cell in a subject. In at least one embodiment of the present disclosure, the SARS-COV-2 is a wild-type or a variant thereof. In at least one embodiment of the present disclosure, the variant is any one selected from the group consisting of Alpha, Beta, Gamma, Delta, and Omicron.

In at least one embodiment of the present disclosure, the non-natural polypeptide inhibits the combination of SARS-COV-2 and a cell in a subject. In at least one embodiment of the present disclosure, the subject cell is a human cell.

In at least one embodiment of the present disclosure, the non-natural polypeptide has at least 75% sequence identity to MHC class I (MHC I) and has an amino acid sequence of SEQ ID NO: 1. In at least one embodiment of the present disclosure, the non-natural polypeptide has at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to MHC I. In at least one embodiment of the present disclosure, the non-natural polypeptide has at least 50%, at least 55%, at least 60%, at least 65%, at least 70%, at least 72%, at least 74%, at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1. In at least one embodiment of the present disclosure, the non-natural polypeptide includes an amino acid sequence of SEQ ID NOs: 2, 3, 4, 5, or 6. In at least one embodiment of the present disclosure, the non-natural polypeptide includes an amino acid sequence that has at least 50%, at least 55%, at least 60%, at least 65%, at least 70%, at least 72%, at least 74%, at least 75%, at least 78%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NOs: 2, 3, 4, 5, or 6. Also provided is a pharmaceutical composition comprising the non-natural polypeptide and a pharmaceutically acceptable carrier thereof.

In other embodiments, the present disclosure provides a use of the above non-natural polypeptides in prevention or treatment of SARS-COV-2 infection. In still other embodiments, the present disclosure provides a use of the above non-natural polypeptides in prevention or treatment of SARS-COV-2 infection by inhibiting binding of SARS-COV-2 with an HLA-C on a cell surface of a cell in a subject in need thereof.

### BRIEF DESCRIPTIONS OF THE DRAWINGS

The present disclosure will become more readily appreciated by reference to the following descriptions in conjunction with the accompanying drawings.

FIG. 1 shows the result of ACE2 protein expressed by Escherichia coli (E. Coli) confirmed by western blotting. M represents marker; A represents ACE2; and the arrow points to ACE2.

FIG. 2 shows the results of spike protein receptor binding domain (wild-type and variants) expressed by E. Coli confirmed by western blotting. M represents marker; Rw represents WT-RBD; Rd represents Delta-RBD; Ro represents Omicron-RBD; and the bands representing each protein were indicated by arrows.

FIG. 3 shows the results of MH-I protein expressed by E. Coli confirmed by western blotting. M represents marker; m represents MH-I; upper arrow points to MHI-mCherry; and lower arrow points to MH-I.

FIG. 4 shows a schematic flow chart of palladium nanofilm electrode (Pd-NTF) modification and antigen conjugation. ITO: indium tin oxide: C₂H₂O₄: oxalic acid.

FIG. 5 is a graph showing the impedance change of WT-RBD and human ACE2 protein conjugation at different concentrations.

FIG. 6 is a graph showing the impedance change of Omicron-RBD (Omi-RBD) and human ACE2 protein conjugation at different concentrations.

FIG. 7 is a graph showing the impedance change of binding of WT-RBD to human ACE2 protein at different MH-I concentrations.

FIG. 8 is a graph showing the impedance change of binding of Delta-RBD to human ACE2 protein at different MH-I concentrations.

FIG. 9 is a graph showing the impedance change of binding of Omicron-RBD to human ACE2 protein at different MH-I concentrations.

FIG. 10 is a graph showing the impedance changes of binding of Omicron (BA.1/BA.2) pseudoviruses to human ACE2 protein at different pseudovirus concentrations.

FIG. 11 shows a bar chart on the effects of MH-I on the binding of Omicron (BA.1) variant to human ACE2 protein.

FIGS. 12A and 12B show the bar charts on the effects of MH-I in inhibiting infection of different variant strains in the spike-pseudovirus neutralization assay. RLU: relative light unit.

FIG. 13 shows a bar chart on the effects of MH-I in inhibiting infection of different variant strains in the SARS-COV-2 plaque reduction assay.

FIG. 14. shows the results of MH-I inhibition on cell entry of different variants of SARS-COV-2 spike pseudotyped lentivirus including wild-type (Wuhan), B.1.1.7, B.1.617.2, BA.1, BA.2, and BA.4/5 into the ACE2-transduced HEK293T (ACE2-293T) cells. The x-axis is the MH-I peptide concentration used, and the y-axis is the percentage of inhibition.

FIGS. 15A and 15B show the results of plaque reduction neutralization test assay on MH-I inhibition of viral infection in Vero E6 cells by different SARS-COV-2 variants including Wuhan, B.1.1.7, BA.1 and BA.4/5 variants.

FIG. 15C is a bar graph showing the results of inhibition of viral infection after pretreatment with 8 μM of MH-I with SARS-COV-2 (BA.4/5) or with Vero E6 cells. ns: not significant.

FIGS. 16A and 16B show the ACE2, TMPRSS2, and HLA-c expression on surface of Jurkat and primary T cells, respectively.

FIGS. 16C and 16D show the result of SARS-COV-2 (Multiplicity of Infection (MOI)=0.25) infection of Jurkat and primary T cells, respectively. The samples were collected 48 h post infection.

FIGS. 16E and 16F show the results of attenuation on infection efficacy of Jurkat and primary T cells by MH-I peptide treatment. MS2: MH-I peptide.

FIG. 17A is a panel of whole-animal luminescence images of mice showing different infectivity of spike pseudovirus in hACE2 transgenic mice.

FIG. 17B shows the whole-animal luminescence images of mice presenting significant reduction in the pulmonary uptake of SARS-COV-2 pseudovirus after pre-treatment of the pseudovirus overexpressing spike protein of B.1.1.7 with MH-I peptides (mice in the lower row) compared to the mice infected with SARS-COV-2-S Luc pseudotyped lentiviruses treated with mCherry protein as control (mice in the upper row).

FIG. 17C is a graph showing the quantification result of the total radiance (photons) using ROI tools in IVIS Series 2000 (PerkinElmer) in the comparison of the pseudovirus pulmonary uptake between mice groups receiving mCherry or MH-I-pretreated pseudovirus (*p<0.05, stats method).

FIG. 18 shows the result of MH-I antibody response assayed by MH-I ELISA after intraperitoneal injection of MH-I in five mice in each treatment group. The mice were injected with either 100 μg purified MH-I or MH-I with adjuvant. The anti-sera titers against MH-I protein were determined using sera collected on days 0, 14, 28, and 35 after the intraperitoneal injection. Each data point represents the mean ±SD. ANOVA was used to analyze the statistical significance. *P<0.05 is considered significant compared with pretreatment (Pre) in the same group.

### DETAILED DESCRIPTIONS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the above examples for carrying out this disclosure without contravening its scope for different aspects and applications.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1% to 50%, 5% to 50%, 10% to 40%, 10% to 20%, or 10% to 15% of the number to which reference is being made.

The term “peptide” used herein refers to a short chain containing more than one amino acid monomers, in which the more than one amino acid monomers are linked to each other by amide bonds. It is to be noted that the amino acid monomers used in the peptide of the present disclosure are not limited to natural amino acids, and the amino acid sequence of the peptide can also include unnatural amino acids, compounds with similar structures, or the deficiency of amino acids.

The terms “polypeptide” and “peptide” are used interchangeably herein to refer to polymers of amino acids of any length. The polymer may be linear or branched. It may comprise modified amino acids and may be interrupted by non-amino acids. The terms also encompass an amino acid polymer that has been modified, e.g., disulfide bond formation, glycosylation, lipidation, acetylation, phosphorylation, or any other manipulation, such as conjugation with a labeling component. The polypeptide can be isolated from natural sources, can be produced by recombinant techniques from a eukaryotic or prokaryotic host, or can be a product of synthetic procedures.

It is understandable that a polypeptide may have a limited number of changes or modifications that may be made within a certain portion of the polypeptide irrelevant to its activity or function and still result in a variant with an acceptable level of equivalent or similar biological activity or function. The term “acceptable level” can mean at least 20%, at least 50%, at least 60%, at least 70%, at least 80%, or at least 90% of the level of the referenced protein as tested in a standard assay as known in the art. Biologically functional variant polypeptides are thus defined herein as those polypeptides in which certain amino acid residues may be substituted. Polypeptides with different substitutions may be made and used in accordance with this disclosure. Modifications and changes may be made in the structure of such polypeptides and still obtain a molecule having similar functions. For example, certain amino acids may be substituted for other amino acids in the peptide/polypeptide structure without appreciable loss of activity. Variants can be prepared according to methods for altering a polypeptide sequence known to one of ordinary skill in the art, such as those are found in references which compile such methods, e.g., “Molecular Cloning: A Laboratory Manual,” J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) Ala, Gly; (ii) Ser, Thr; (iii) Gln, Asn; (iv) Glu, Asp; (v) Met, Ile, Leu, Val; (vi) Phe, Tyr, Trp; and (vii) Lys, Arg, His.

Peptides used herein may be isolated from a variety of sources, such as from human tissue types or from other sources, or prepared by recombinant or synthetic methods, or by any combination of these and similar techniques. Peptide variants include peptides comprising amino acid sequences sufficiently identical to or derived from the amino acid sequence of a native peptide which includes fewer amino acids than the native peptides. A portion or a fragment of a peptide can be a peptide which is, for example, 3 to 5, 8 to 10, 10, 15, 15 to 20, 20, 25, 30, 35, 40, 45, 50, 60, 70, 80, 90, 100, 110, 120, 130, 150, 200, 300 or more amino acids in length. Portions or fragments in which regions of a polypeptide are deleted can be prepared by recombinant techniques and can be evaluated for one or more functional activities such as the ability to form antibodies specific to a peptide. A portion or a fragment of a peptide may comprise a domain of the native peptide or a portion or a fragment of such domain.

As used herein, the term “sequence identity” or, for example, comprising a “sequence having 80% sequence identity with,” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 80%, about 85%, about 90%, about 95%, about 97%, about 98%, about 99% or about 100% sequence identity to any of the reference sequences described herein (see, e.g., Sequence Listing), typically where the polypeptide variant maintains at least one biological activity or function of the reference polypeptide.

The term “detect,” “detecting,” or “detection” includes assaying, or otherwise establishing the presence or absence of the target molecule(s), protein domain(s), subunits, or combinations of reagent-bound targets, and the like.

The terms “subject,” “patient,” and “individual” are used interchangeably herein and refer to a warm-blooded animal such as a mammal that is afflicted with, or suspected of having, at risk for or being pre-disposed to, or being screened for viral infection, including actual or suspected SARS-COV-2 infection. These terms include, but are not limited to, domestic animals, sports animals, primates, and humans. For example, the terms refer to a human.

As used herein, the terms “therapies” and “therapy” can refer to any protocol(s), method(s), composition(s), formulation(s), and/or agent(s) that can be used in prevention or treatment of a disease or symptom associated therewith. In at least one embodiment, the terms “therapies” and “therapy” refer to biological therapy, supportive therapy, and/or other therapies useful in prevention or treatment of a disease or symptom associated therewith known to one of ordinary skill in the art.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen) to delay or prevent disease occurrence. Treatment may also be continued after symptoms have resolved, for example, to delay or prevent recurrence.

As used herein, the term “preventing” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease. The preventive measures are used to avoid, prevent, or postpone the occurrence of the disease or the symptoms or conditions of the disease.

The kits provided herein are in suitable packaging. Suitable packaging includes, but is not limited to, vials, bottles, jars, flexible packaging, and the like. Also contemplated are packages for use in combination with a medical device, such as an inhaler, nasal administration device, or an infusion device. A kit may have a sterile access port (for example, the container may be an intravenous solution bag or a vial having a stopper pierceable by a hypodermic injection needle). The container may also have a sterile access port. Kits optionally may provide additional components such as buffers and interpretive information. Normally, the kit comprises a container and a label or package insert(s) on or associated with the container. In some embodiments, the disclosure provides articles of manufacture comprising contents of the kits described above.

As used herein, the term “pharmaceutical composition” or “pharmaceutical combination” can be prepared according to any method known to the art for the manufacture of pharmaceuticals. Such composition or combination may contain sweetening agents, flavoring agents, coloring agents, and preserving agents. A formulation can be admixed with nontoxic and pharmaceutically acceptable excipients which are suitable for manufacture. Non-limiting formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, lozenges, packets, troches, elixirs, suspensions, solutions, syrups, soft and hard gelatin capsules, suppositories, sterilized injection fluid, packaged powder, on patches, in implants, etc.

As used herein, pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; physiological saline; sterilized water; isotonic agents; and/or non-ionic surfactants. See, e.g., Remington: The Science and Practice of Pharmacy 20^{th} Ed. (2000) Lippincott Williams and Wilkins, Ed. K. E. Hoover.

### EXAMPLES

Exemplary embodiments of the present disclosure are further described in the following examples, which should not be construed to limit the scope of the present disclosure.

In the following embodiments, the electrochemical palladium nanofilm electrode (Pd-NTF) functionalized with angiotensin-converting enzyme II (ACE2) was used as the electrode probe of the electrochemical impedance spectrometer (EIS) to analyze the interaction relationship between ACE2 and the receptor binding domain (RBD) of spike protein in wild type or mutant virus.

The MH-I protein used in this example includes a fragment of major histocompatibility complex (MHC) class I (MHC I) and has an amino acid sequence of SEQ ID NO: 1.

### Example 1. Preparation of ACE2 Protein, Receptor Binding Domain (RBD) of Wild Type/Mutant SARS-COV-2 Spike Protein and MH-I Protein

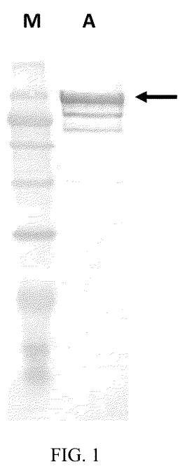

The ACE2 peptides were recombinantly expressed by Escherichia coli (E. coli) using the bacterial protein expression method well-known by a person skilled in the art and then purified. The identity and purity of the expressed proteins were confirmed by western blotting analysis using anti-ACE2 antibodies (FIG. 1). The expressed target protein ACE2 has a full length of 805 amino acids, a molecular weight of about 92.5 kDa, and an isoelectric point of 5.55.

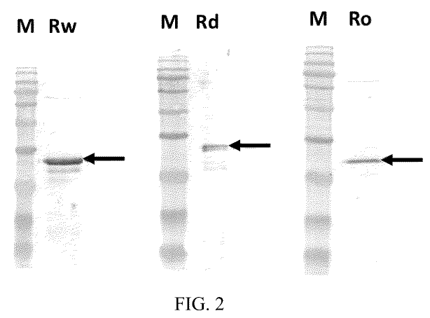

The receptor binding domains (RBD) of wild type, Delta, and Omicron (BA.1) SARS-COV-2 spike proteins (WT-RBD, Delta-RBD, and Omicron-RBD) were expressed by E. Coli using the bacterial protein expression method well-known by a person skilled in the art and then purified. The identity and purity of the expressed proteins were confirmed by western blotting analysis by anti-spike protein antibodies (FIG. 2). The expressed target protein WT-RBD has a full length of 224 amino acids, a molecular weight of about 25.3 kDa, and an isoelectric point of 8.12; the expressed target protein Delta-RBD has a full length of 217 amino acids, a molecular weight of about 24.9 kDa, and an isoelectric point of 8.34; and the expressed target protein Omicron-RBD has a full length of 209 amino acids, a molecular weight of about 24 kDa, and an isoelectric point of 8.64.

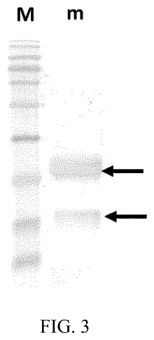

Red fluorescent protein (mCherry) was added to the c-terminus of the MH-I peptide in this example to stabilize it, and the end of MHI-mCherry was connected to a segment of 6 consecutive amino acid (histidine) sequences. The expressed proteins were confirmed by western blotting analysis (FIG. 3). The MHI-mCherry peptides were expressed by E. Coli using the bacterial protein expression method well-known by a person skilled in the art and then purified with polyhistidine (His)-Tag. The expressed target protein MHI-mCherry has a full length of 359 amino acids.

### Example 2. Binding Test of ACE2 Peptide Towards SARS-COV-2 Spike Protein

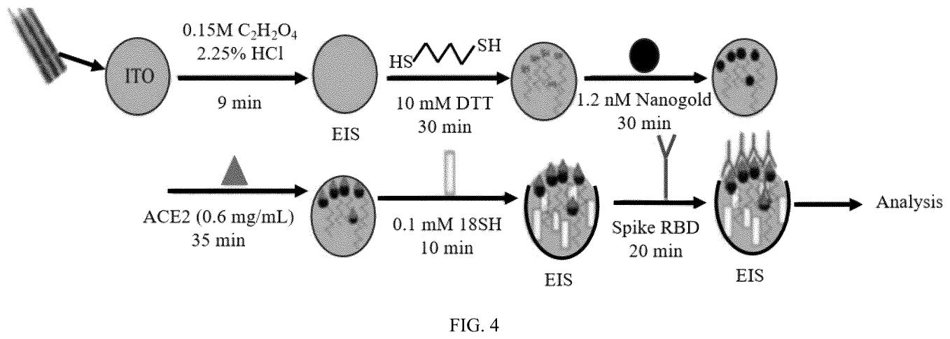

Previous studies have pointed out that SARS-COV-2 binds to human ACE2 protein to infect humans, so this example simulates the way that SARS-COV-2 infects human and uses human ACE2 protein to functionalize electrochemical electrodes. In this example, a surface-active electrochemical palladium nanofilm electrode (Pd-NTF) previously developed by the inventor was used (Kiew et. al., Biosens. Bioelectron., 2021). The electrode was activated with 2 μL dithiothreitol (DTT) (10 mM) and 2 μL nanogold (1.2 nM) first, so that the electrode could more stably bind 2 μL human ACE2 protein (0.6 mg/mL) on the surface of the palladium electrode to increase the impedance. Then, the electrode surface gap was filled with octadecanethiol (18SH) for saturation to complete the preparation of the ACE2-modified functionalized electrode. Afterwards, electrochemical impedance spectroscopy (EIS) was used to analyze the binding ability of spike protein RBD of wild type SARS-COV-2 and SARS-COV-2 of different mutants to human ACE2 protein (FIG. 4). Using the ratio of impedance changes generated by the binding of spike protein RBD of wild-type or different mutants to human ACE2 protein conjugated to the palladium electrode, such as equation (1), the binding ability of different spike protein RBDs to human ACE2 protein can be analyzed.

$$Δ {R}_{ct} = ( {R}_{ct - RBD} - {R}_{ct - ACE 2} ) / {R}_{ct - ACE 2} \qquad ( Equation 1 )$$

In equation 1, R_{ct-RBD} refers to the charge transfer impedance (R_{ct}) of RBD; R_{ct-ACE2} refers to the charge transfer impedance of ACE2; and ΔR_{ct} refers to the changes of charge transfer impedance.

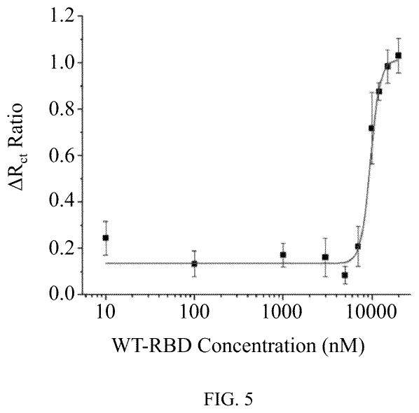

For example, EIS analysis of the conjugation of ACE2 with WT-RBD was carried out. The impedance changes generated by the conjugation of WT-RBD at different concentrations (0.01 μM to 20 μM) with the ACE2-functionalized palladium electrode showed that the impedance change began to increase when the concentration of WT-RBD was 7 μM, and the reaction reaches saturation at a concentration of 20 μM (FIG. 5). After fitting and calculating with the Hill 1 equation as shown in Table 1, it can be seen that the equivalent dissociation constant (k_{d}) of WT-RBD binding to human ACE2 protein is 9.44±0.78 μM. The detailed fitting results and comparisons are shown in Table 1.

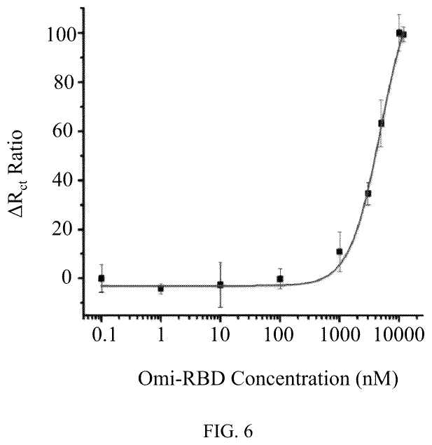

Further, EIS analysis of the conjugation of ACE2 with Omicron-RBD was also carried out. The impedance changes generated by the conjugation of Omicron-RBD at different concentrations (0.1 μM to 20 μM) with the ACE2-functionalized palladium electrode showed that the impedance change began to increase when the concentration of Omicron-RBD was 1 μM, and the reaction reaches saturation at a concentration of 10 μM (FIG. 6). After fitting and calculating with the Hill 1 equation as shown in Table 1, it can be seen that the equivalent dissociation constant (k_{d}) of Omicron-RBD binding to human ACE2 protein is 4.32±0.3 μM. The detailed fitting results and comparisons are shown in Table 1.

TABLE 1
Analysis parameters of the Hill 1 equation for the ability of different
SARS-CoV-2 spike protein RBDs to bind to human ACE2 protein

| | |
|---|---|
| WT-RBD | Omicron-RBD |

| | |
|---|---|
| Model | Hill 1 |
| Equation | y = START + (END − START) × xⁿ/(kⁿ + xⁿ) |

| | | |
|---|---|---|
| V₀ | 0.13 | 0.29 |
| Vₘₐₓ | 0.97 | 1.57 |
| k_{d} | 9.44 ± 0.78 μM | 4.32 ± 0.3 μM |

These results show that EIS analysis of ACE2-coated Pd-NTF electrode is a sensitive method to evaluate conjugation between ACE2 and other proteins.

### Example 3. Analysis of the Effect of Functional Protein MH-I on the Binding of Spike Protein RBD to Human ACE2 Protein

In this example, after the MH-I protein of the present disclosure was bound to the spike protein RBD of wild-type or different mutants, changes in the binding abilities of spike protein RBD of wild-type or different mutants with human ACE2 protein conjugated to the palladium electrode were observed.

In this example, different concentrations of MH-I were mixed with spike protein RBD of wild-type or different mutants for 30 minutes to obtain mixtures of MH-I and spike protein RBD of wild-type or different mutants (MH-I-S-RBD) at increasing MH-I concentrations (0.01 to 1 mg/mL), and then 2 μL of each mixture was added to the ACE2-modified functionalized electrode surface for 20 minutes. EIS was carried out to measure impedance changes. Using the ratio of impedance changes generated by the binding of MH-I-S-RBD to human ACE2 protein conjugated to the palladium electrode, such as equation (2), calculated by subtracting the baseline R_{ct} signals of the ACE2-Pd-NTF electrode from those of the electrodes treated with the protein mixtures, the extent that MH-I interferes the ability of spike protein RBD to bind human ACE2 protein can be analyzed.

$$Δ {R}_{ct} = ( {R}_{ct - MH ‐ I - S ‐ RBD} - {R}_{ct - ACE 2} ) / {R}_{ct - ACE 2} \qquad ( Equation 2 )$$

In equation 2, R_{ct-MH-I-S-RBD} refers to the charge transfer impedance (R_{ct}) of MH-I-S-RBD; R_{ct-ACE2} refers to the charge transfer impedance of ACE2; and ΔR_{ct} refers to the changes of charge transfer impedance.

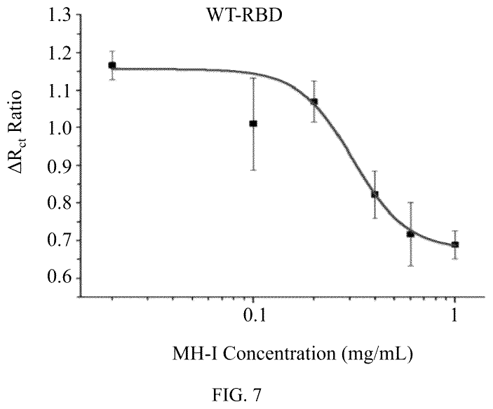

For example, EIS analysis of the effect of MH-I on the conjugation of ACE2 with WT-RBD was carried out. In this example, EIS was used to analyze the effect of different MH-I concentrations (0.02 mg/mL to 1 mg/mL) on the binding ability of WT-RBD to human ACE2 protein. It can be seen from FIG. 7 that the impedance change ratio at 0.02 mg/mL of MH-I was 1.16, and with the increase of MH-I concentration, the ratio of impedance changes gradually decreased to 0.68. In other words, MH-I has the function of interfering with the binding reaction between WT-RBD and human ACE2 protein. The detailed fitting results and the effect of MH-I on the conjugation of ACE2 with different RBDs are shown in Table 2.

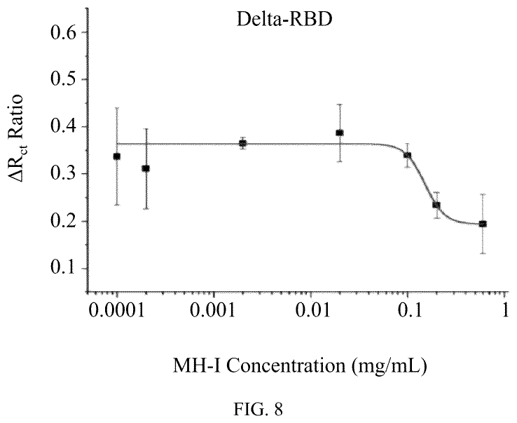

Further, EIS analysis of the effect of MH-I on the conjugation of ACE2 with Delta-RBD was carried out. In this example, EIS was used to analyze the effect of different MH-I concentrations (0.1 μg/mL to 0.6 mg/mL) on the binding ability of Delta-RBD to human ACE2 protein. It can be seen from FIG. 8 that the impedance change ratio at 0.1 μg/mL MH-I was 0.34, and with the increase of MH-I concentration, the ratio of impedance changes gradually decreased to 0.19. In other words, MH-I has the function of interfering with the binding reaction between Delta-RBD and human ACE2 protein. The detailed fitting results and the effect of MH-I on the conjugation of ACE2 with different RBDs are shown in Table 2.

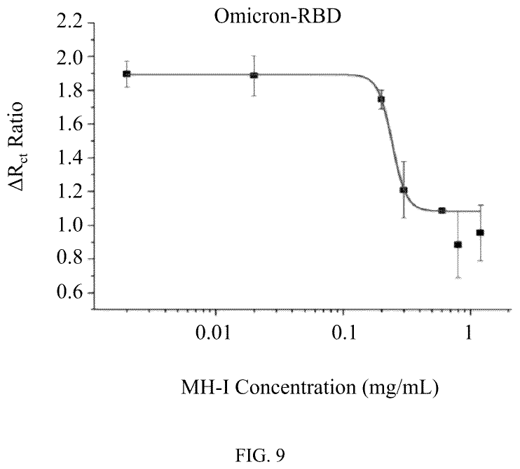

Also, EIS analysis of the effect of MH-I on the conjugation of ACE2 with Omicron-RBD was carried out. In this example, EIS was used to analyze the effect of different MH-I concentrations (2 μg/mL to 1.2 mg/mL) on the binding ability of Omicron-RBD to human ACE2 protein. It can be seen from FIG. 9 that the impedance change ratio at 2 μg/mL MH-I was 1.89, and with the increase of MH-I concentration, the ratio of impedance changes gradually decreased to 0.95. In other words, MH-I has the function of interfering with the binding reaction between Omicron-RBD and human ACE2 protein. The detailed fitting results and the effect of MH-I on the conjugation of ACE2 with different RBDs are shown in Table 2.

TABLE 2
Analysis parameters of the Hill 1 equation for
the effect of MH-I on the binding of ACE2 with
different SARS-CoV-2 spike protein RBDs

| Spike RBD type | WT-RBD | Delta-RBD | Omicron-RBD |
|---|---|---|---|
| Model | Hill 1 | | |
| Equation | y = START + (END − START) × xⁿ/(kⁿ + xⁿ) | | |
| EC₅₀ | 0.31 ± 0.04 | 0.15 ± 0.09 | 0.24 ± 0.02 |
| | mg/mL | mg/mL | mg/mL |

These results demonstrate the capability of MH-I to bind to free spike protein RBD of all variants and attenuate their interactions with the immobilized ACE2. This further suggests the possibility of using MH-I to interfere with SARS-COV-2 cell entry, thereby attenuating the progression of infection.

### Example 4. Analysis of ACE2 Binding to Omicron Variant Pseudoviruses

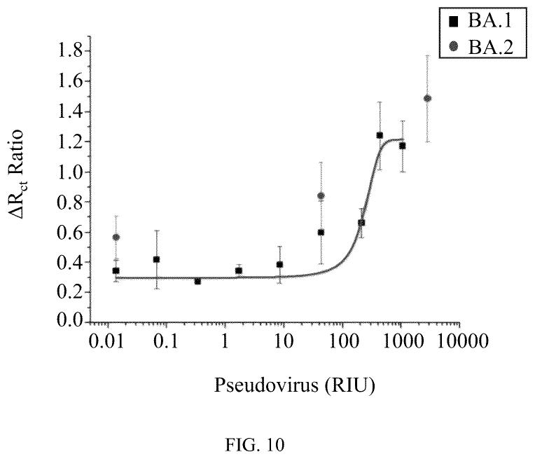

In this example, pseudoviruses of Omicron BA.1 and BA.2 variants were further used to test the EIS analysis by ACE2-functionalized palladium electrode as a tool to analyze binding between ACE2 and viruses. The graph on impedance changes generated by the binding of Omicron (BA.1) variant pseudovirus at different concentrations (0.0137 relative infection unit (RIU) to 1,070 RIU) (shown as squares in FIG. 10) with the ACE2-functionalized palladium electrode shows that when the concentration of the Omicron (BA.1) variant is 42.8 RIU, the impedance change begins to increase, and the binding reaches saturation at the concentration of 1,070 RIU. After fitting and calculating with Boltzmann equation, it can be seen that the equivalent dissociation constant of Omicron (BA.1) binding to human ACE2 protein is 231±449 RIU.

The graph on impedance changes generated by the binding of Omicron (BA.2) variant pseudovirus at different concentrations (0.0137 RIU, 42.8 RIU, and 2,817 RIU) (shown as circles in FIG. 10) with the ACE2-functionalized palladium electrode shows that the ratio of impedance changes of Omicron (BA.2) binding to the human ACE2 protein is larger than that of Omicron (BA.1). Thus, it can be seen that the binding ability of Omicron (BA.2) to the human ACE2 protein is higher than that of Omicron (BA.1).

Example 5. Analysis of the Effect of MH-I on the Conjugation of ACE2 with Omicron (BA.1) Variant Pseudovirus

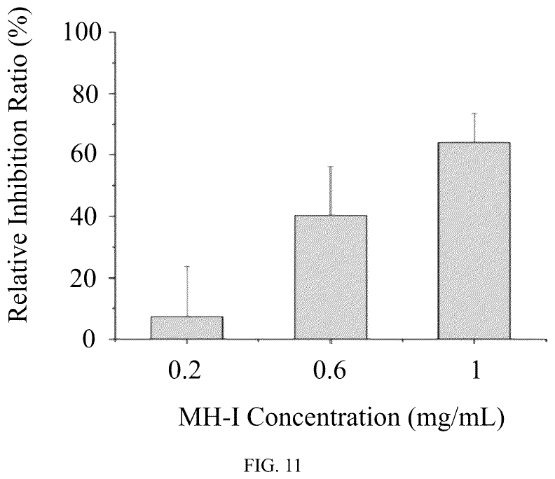

In this example, EIS was used to analyze the effect of MH-I on the binding of Omicron (BA.1) to human ACE2 protein at different concentrations (0.2 mg/mL, 0.6 mg/mL, and 1 mg/mL). It can be seen from FIG. 11 that when 0.2 mg/mL of MH-I was added, the binding between Omicron (BA.1) and human ACE2 protein was inhibited by about 14.12%, and when the MH-I concentration was increased to 0.6 mg/mL, the inhibitory effect could reach 40.36%. Further, when the MH-I concentration was increased to 1 mg/mL, the binding of Omicron (BA.1) and human ACE2 protein was inhibited by 64.02%. It thus can be seen that MH-I has the function of interfering with the binding reaction of Omicron (BA.1) variant pseudovirus and human ACE2 protein.

### Example 6. Analysis of the Effects of MH-I in Inhibiting Virus Infection of SARS-COV-2 Viruses

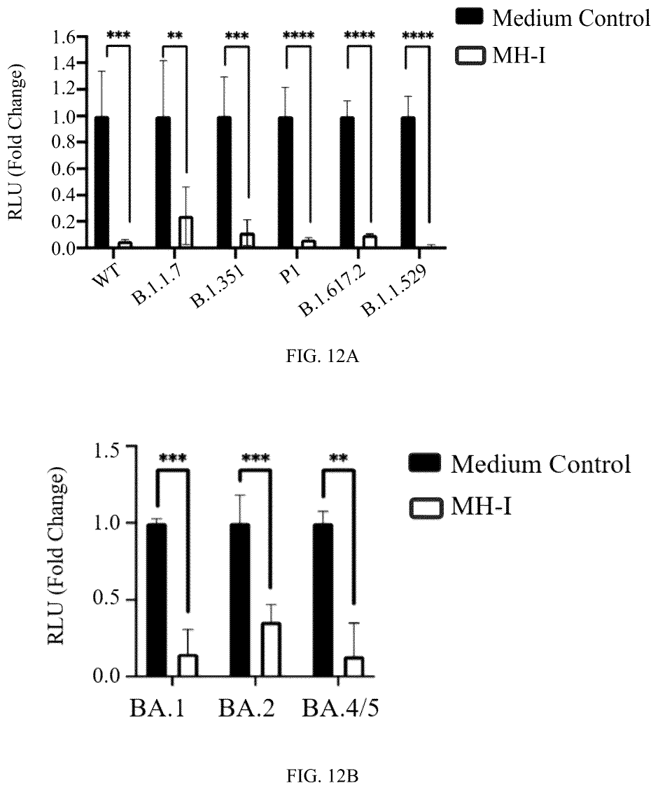

In this example, MH-I (2.1 μM) was pre-incubated with different spike-variant pseudoviruses (9,000 RIU) for 1 hour at 37° C. and later used to infect 293T-ACE2 cells for 48 hours. Infectivity was measured by Bright-Glo Luciferase assay system following the instruction manual. A culture medium control group was used to normalize the results. It was shown that MH-I can inhibit the infection of different spike-variant pseudoviruses, including WT, B.1.1.7, B1.351, P1, B.1.617.2, B.1.1.529, BA.1, BA.2, and BA.4/5 (FIGS. 12A and 12B).

To analyze the IC₅₀ of MH-I against the SARS-COV2 virus, 100 TCID50 (50% tissue culture infectious dose) of SARS-COV-2/human/TWN/CGMH-CGU-01/2020 virus were pre-incubated with different concentrations of MH-I at 37° C. for 1 hour before infecting Vero-TMPRSS2 (transmembrane serine protease 2). After 5 days of post-infection, the virus was inactivated with 100 μL of 10% formalin for at least 1 hour, followed by staining the cell with 0.5% crystal violet. After calculation, the IC₅₀ of MH-I against the SARS-COV-2 (WT) virus was 3.32±0.41 μM, and that of mCherry (control group) was greater than 7.7 μM, indicating that MH-I can specifically inhibit SARS-COV-2 (WT) virus infection.

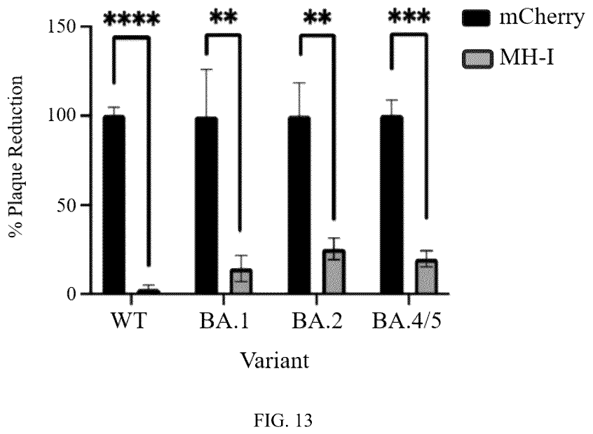

In addition, the inhibitory effect of MH-I on different SARS-COV-2 variants was analyzed by plaque reduction assay. MH-I (10 μM) was co-incubated with 50 p.f.u. (plaque-forming unit) of different SARS-COV-2 variants comprising SARS-COV-2/human/TWN/CGMH-CGU-01/2020 (WT), SARS-COV-2/human/TWN/CGMH-CGU-90/2021 (BA.1), SARS-COV-2/human/TWN/CGMH-CGU-158/2022 (BA.2), and hCoV-19/Taiwan/689423/2022 (BA.4/5), followed by infecting Vero E6 cells with these variants at 35° C. After 1 hour, the Vero E6 cells were washed once with phosphate buffered saline (PBS) and added with 3 mL of E-0 DMEM (Dulbecco's Modified Eagle Medium, containing 1.2% microcrystalline cellulose (MCC)). After 72 hours, cells were inactivated by PBS washing and stained with 0.5% crystal violet (containing 10% formalin). The result shows that MH-I could significantly inhibit the WT, BA.1, BA.2, and BA.4/5 variants (FIG. 13).

### Example 7. MH-I Inhibits SARS-COV-2 Virus Infection

The effects of MH-I on SARS-COV-2 infection in vitro were further assessed by performing a neutralization assay using pseudoviruses expressing different spike protein variants.

Briefly, wildtype (Wuhan) or variants of SARS-COV-2-S Luc pseudotyped lentiviruses were prepared in 293T cells. The vectors pcDNA3.1, pCMVdeltaR8.91, and pLAS2w.FLuc.Ppuro were used to express the spike protein on the viral surface, after which the virus was selected using puromycin to quantify the titers. The SARS-COV-2-S Luc pseudotyped lentivirus was provided by the National RNAi Core Facility (Academia Sinica, Taiwan). The SARS-COV-2-S Luc pseudotyped lentivirus exhibited spike protein mutations shown in Table 3.

TABLE 3
Spike protein mutations of SARS-CoV-
2-S Luc pseudotyped lentiviruses

| Variants | Mutations in Spike |
|---|---|
| B.1.1.7 | 69-70 del, Y144 del, N501Y, A570D, D614G, P681H, T716I, |
| | S982A, D1118H |
| B.1.617.2 | T19R, G142D, 156-157 del, R158G, L452R, T478K, D614G, |
| | P681R, D950N |
| BA.1 | A67V, 69-70 del, T951, G142D, 143-145 del, 211 del, L212I, |
| | ins214EPE, G339D, S371L, S373P, S375F, K417N, N440K, |
| | G446S, S477N, T478K, E484A, Q493R, G496S, Q498R, |
| | N501Y, Y505H, T547K, D614G, H655Y, N679K, P681H, |
| | N764K, D796Y, N856K, Q954H, N969K, L984F |
| BA.2 | L24S, 25-27 del, T91I, G142D, V213G, G339D, S371F, |
| | S373P, S375F, T376A, D405N, R408S, K417N, N440K, |
| | S477N, T478K, E484A, Q493R, Q498R, N501Y, Y505H, |
| | D614G, H655Y, N679K, P681H, N764K, D796Y, Q954H, |
| | N969K |
| BA.4/5 | L24S, 25-27 del, 69-70del, T91I, G142D, V213G, G339D, |
| | S371F, S373P, S375F, T376A, D405N, R408S, K417N, |
| | N440K, L452R, S477N, T478K, E484A, F486V, Q498R, |
| | N501Y, Y505H, D614G, H655Y, N679K, P681H, N764K, |
| | D796Y, Q954H, N969K |

The ACE2-transduced HEK293T (ACE2-293T) cells were maintained in Dulbecco's minimal essential medium (DMEM) supplemented with 10% (v/v) FBS and 10 μg/mL blasticidin. Approximately 40,000 cells/well were seeded in a 96-well plate the day before the experiment, and the cells were approximately 80% confluent.

The Wuhan or variant SARS-COV-2 (B.1.1.7, B.1.617.2, BA.1, BA.2, and BA.4/5) spike pseudotyped lentiviruses containing 9,000 RIU (relative infection unit) were pre-incubated with different dilutions of MH-I in triplicates for 1 h at 37° C. Then, 100 μL of the MH-I virus mixture was transferred to the cells. At 24 h post-infection, the medium was removed from the wells, and 100 μL of 10% FBS in DMEM were added. Afterwards, at 48 h post-infection, 50 μL of the medium was removed, and 50 μL of Bright-Glo substrate (E2610, Promega, Madison, WI, USA) was added to induce cell lysis. The luminescence signal was measured using a Synergy 2 microplate reader (BioTek, Winooski, VT, USA).

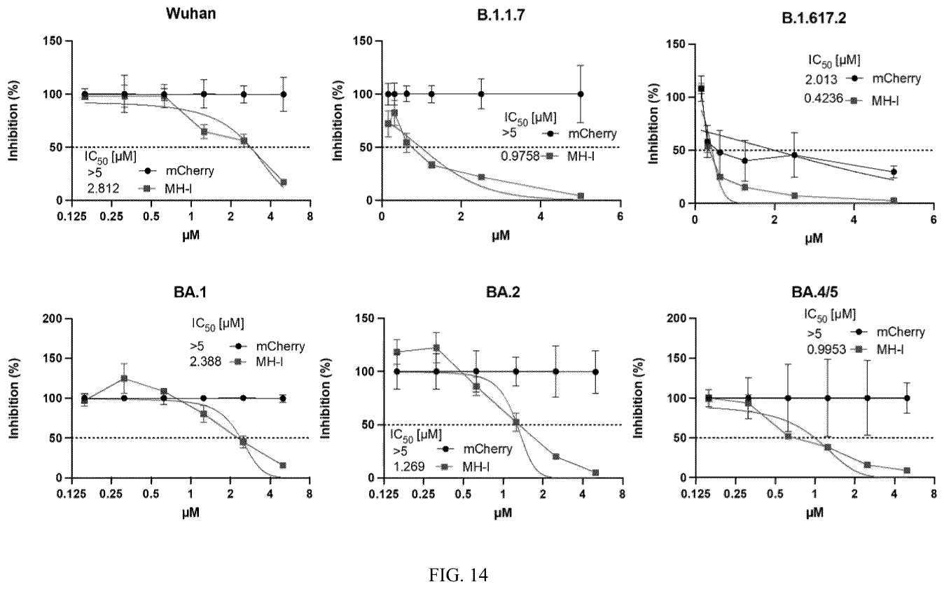

These experiments revealed that MH-I effectively inhibited the entry of various SARS-COV-2 pseudotyped lentivirus strains including wild-type (Wuhan), B.1.1.7, B.1.617.2, BA.1, BA.2, and BA.4/5 Omicron variants into ACE2-transduced HEK293T cells. This suppression was evident from a significant concentration-dependent reduction in the luminescence signals detected in the lysates of cells treated with the MH-I-pseudovirus mixture. As the MH-I of this disclosure incorporates the mCherry tag, it is used as a control protein for comparison. It was found that the depletion levels for Wuhan, B.1.1.7, B.1.617.2, BA1, BA.2, and BA.4/5 variants were 82.7%±0.58, 95.7%±0.58, 97.3%±0.58, 84.3%±1.15, 95%±0.0, and 91%±1.0, respectively, at the maximum MH-I treatment dose of 5 μM (FIG. 14).

Furthermore, the 50% inhibitory concentration (IC₅₀) values of MH-I against pseudovirus strains expressing the spike protein variants Wuhan, B.1.1.7, B.1.617.2 BA1, BA.2, and BA.4/5 were determined to be 2.812, 0.9758, 0.4236, 2.388, 1.269, and 0.9953, respectively. In contrast, the entry of the pseudovirus into ACE2-293T cells was not affected when the viruses were incubated with the control protein (mCherry; FIG. 14). However, mCherry also showed a notable inhibitory effect on the pseudovirus carrying the spike protein of the B.1.617.2 variant; therefore, this inhibitory effect against the B. 1.617.2 variant could not be solely attributed to MH-I.

To assess the efficacy of MH-I against the real SARS-COV-2 virus, plaque reduction neutralization test (PRNT) assay was performed. Briefly, Vero E6 cells were seeded at 1.2×10⁶ cells/well in a 6-well plate and incubated at 35° C. for 24 h. Then, 50 plaque-forming unit (PFU) of the virus were pre-incubated with different concentrations of the MH-I peptide and added to the cells.

For the plaque reduction assay, the cells were pretreated with different concentrations of MH-I peptide. After 1 h of adsorption, the cells were washed with PBS and supplemented with 3 mL of 1.2 percent microcrystalline cellulose (MCC; Sigma 435244) in serum-free DMEM. The cells were incubated at 37° C. for 72 h and fixed with 10% formaldehyde. The cells were stained with crystal violet, and viral plaques were counted and calculated as the number of PFU per milliliter.

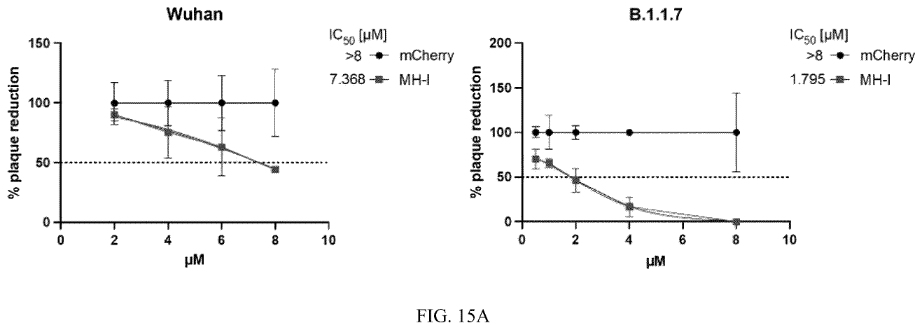

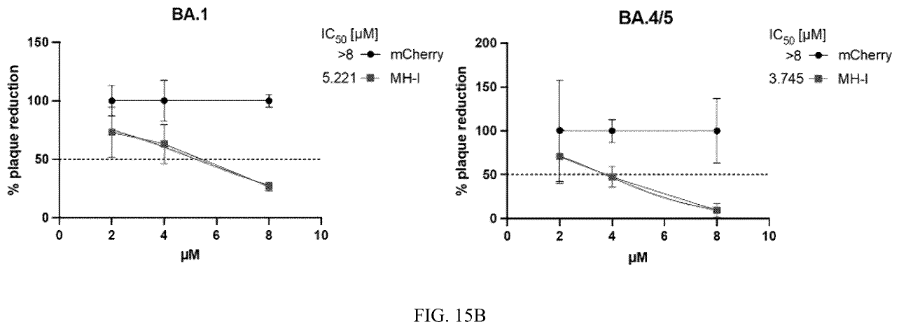

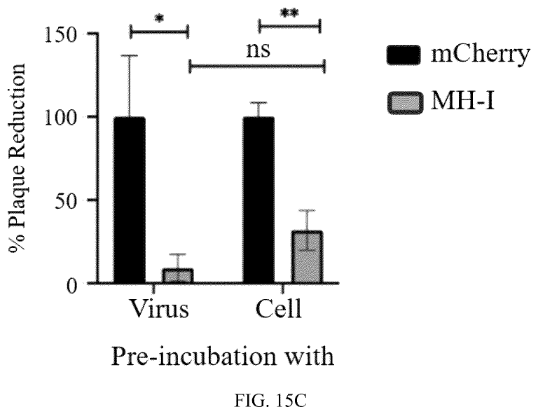

In the MH-I pretreatment experiment (FIG. 15C), the MH-I peptide was pre-mixed with SARS-COV-2 or Vero E6 cell and incubated at 35° C. for 1 h. Next, either the MH-I-SARS-COV-2 mixture or SARS-COV-2 alone was added to the Vero E6 cells and incubated at 35° C. for 1 h. After a 1-hour adsorption period, the Vero E6 cells were washed and overlaid with microcrystalline cellulose (MCC), following the same protocol described earlier.

As a result, MH-I demonstrated significant inhibition of SARS-COV-2 infection by variants of Wuhan, B.1.1.7, BA.1, and BA.4/5 at the concentration of 8 μM with over 50% plaque reduction, while 100% plaque reduction were observed in Vero E6 cells treated with B.1.1.7 and BA.4/5 variants (FIGS. 15A and 15B). Moreover, pretreatment of the virus or cells with MH-I substantially inhibited viral infection (FIG. 15C).

### Example 8. SARS-COV-2 Entered T Cells Through HLA-C

MH-I peptide derives from HLA-C and exhibits a strong binding ability to the RBD, and it is shown in the above examples that MH-I peptide effectively blocks virus entry into cells. In this example, HLA-C is shown as an alternative route for SARS-COV-2 infection in certain cells that show weak or no expression of ACE2 and TMPRSS2 but high levels of HLA-C.

In this example, Jurkat cells and primary T cells were used as the cell models of SARS-COV-2 infection. Jurkat cells were purchased from the Bioresource Collection and Research Center (BCRC, Hsinchu, Taiwan) and maintained in RPMI 1640 growth medium (Gibco/Invitrogen) supplemented with heat-inactivated 10% fetal bovine serum (FBS, HyClone) and 1% penicillin/streptomycin (Gibco/Invitrogen). Primary T cells were isolated from fresh peripheral blood mononuclear cells (PBMCs) using an Easy Sep Human T Cell Isolation Kit (STEMCELL Technologies, negative selection) according to the manufacturer's instructions.

First, the expression patterns of ACE2, TMPRSS2, and HLA-C on the surfaces of the Jurkat and primary T cells were analyzed. Briefly, Jurkat or primary T cells were suspended in the staining buffer (PBS containing 1% skim milk) at a density of 2×10⁵ and incubated with anti-ACE2 (R&D Systems), anti-TMPRSS2 (Santa Cruz), and anti-HLA-C (Novus Biologicals) antibodies at 4° C. for 30 min. After washing with PBS, 1 μg of FITC-conjugated secondary antibody (Thermo Scientific, Waltham, MA, USA and Jackson Immunoresearch, West Grove, PA, USA) was added and incubated at 4° C. for 30 min. T-cell expression was analyzed using a flow cytometer (BD Accuri C6)

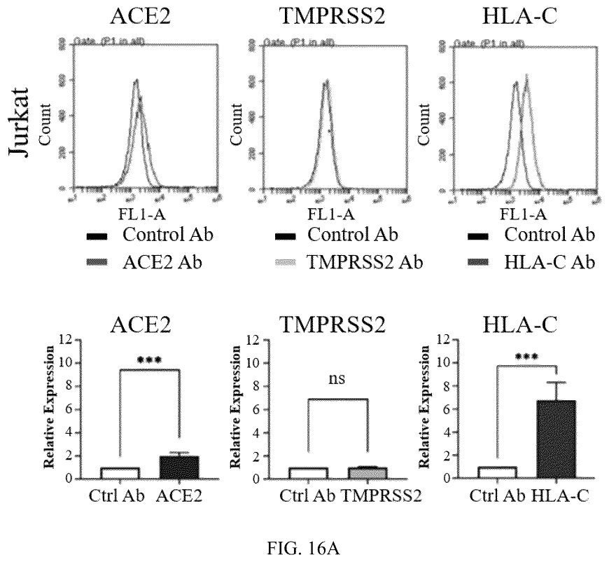

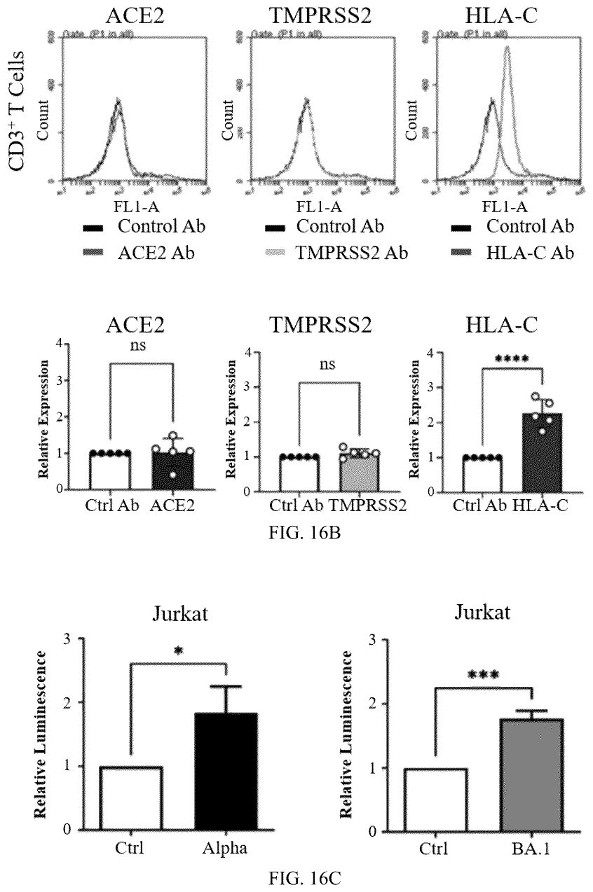

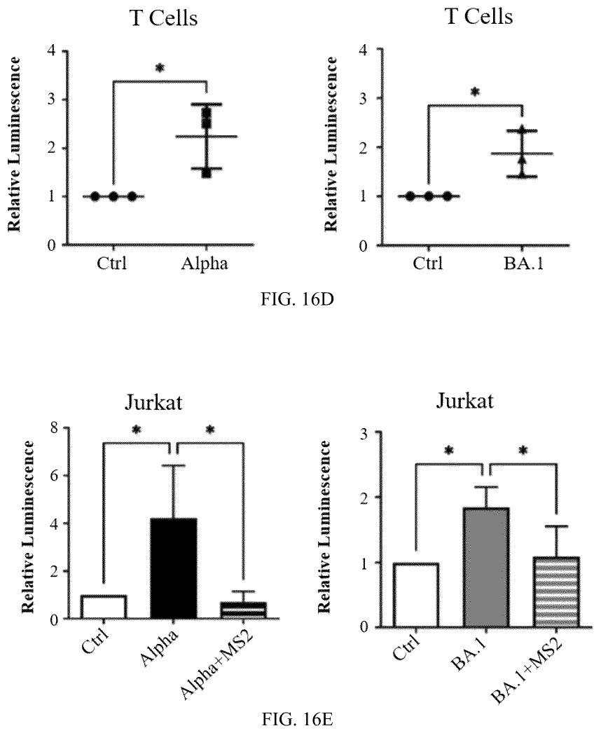

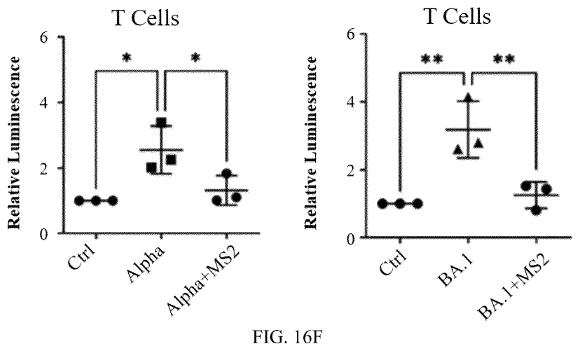

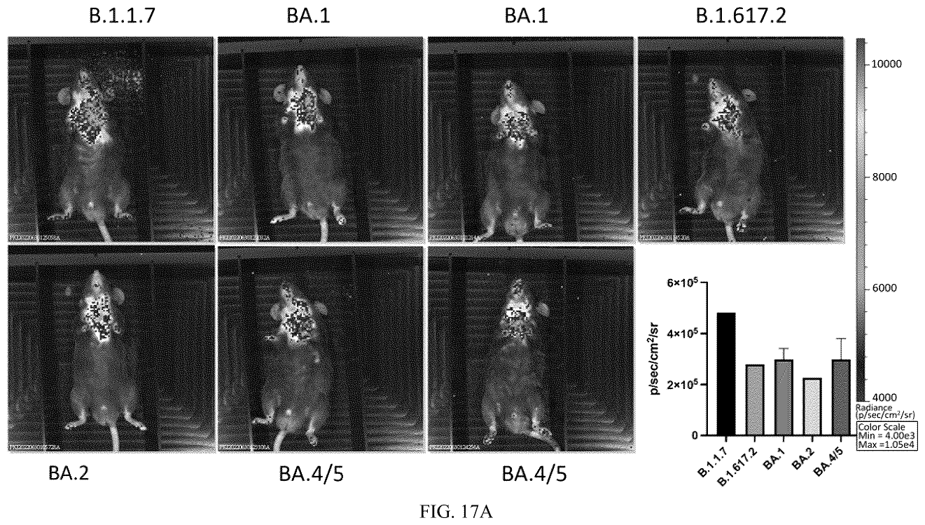

The results showed that the Jurkat cells weakly expressed ACE2 and highly expressed HLA-C but did not express TMPRSS2 (FIG. 16A). Primary T cells highly expressed HLA-C but not ACE2 or TMPRSS2 (FIG. 16B).

When Jurkat or T cells were treated with pseudotyped lentiviruses carrying SARS-COV-2 spike proteins of the B.1.1.7 Alpha and BA.1 Omicron variants, both Jurkat and primary T cells were infected. To carry out the experiment, SARS-COV-2 spike (B.1.1.7 Alpha and BA.1 Omicron variants) pseudotyped lentivirus were purchased from RNAi Core (Academia Sinica, Taipei, Taiwan) and used to detect T-cell infection. Jurkat cells or primary T cells were seeded with 10⁴ cells/well in a 96-well microculture plate, to which the spike (Alpha or BA.1) pseudotyped lentivirus (MOI=0.25) was added and incubated for 24 h. The culture medium was replaced with fresh growth medium, and the cells were incubated for an additional 24 h. The infective cells were collected and added to a white plate, to which culture medium supplemented with D-luciferin (Perkin Elmer, Waltham, Mass, USA) was added to a final concentration of 150 mg/mL. Luminescence was measured using a BioTek Synergy H1 microplate reader.

The results that both Jurkat and primary T cells were infected indicated that SARS-COV-2 could enter T cells in the absence of ACE2 and TMPRSS2 (FIGS. 16C and 16D).

To confirm that HLA-C is a possible pathway for SARS-COV-2 entry into T cells, MH-I peptides were used to block the binding of the SARS-COV-2 spike protein to HLA-C on T cells. Jurkat cells (10⁴ cells/well) were seeded in 96-well microplates, and MH-I peptide was added simultaneously to each spiked pseudotyped lentivirus (MOI=0.25) and incubated for 24 h. The culture medium was replaced with fresh growth medium, and the cells were incubated for an additional 24 h. The cells were collected and added to a white plate, to which culture medium supplemented with D-luciferin (Perkin Elmer, Waltham, Mass, USA) was added to obtain a final concentration of 150 mg/mL. Luminescence was measured using a luminescence filter on a BioTek Synergy H1 microplate reader.

The results show that MH-I peptide effectively blocked the response to SARS-COV-2 infection in Jurkat and primary T cells (FIGS. 16E and 16F), indicating that HLA-C acts as one of the routes that SARS-COV-2 adopts for entry into T cells.

### Example 9. MH-I Attenuates SARS-COV-2 Pseudovirus Pulmonary Uptake In Vivo

In vivo virus inhibition capability of MH-I using whole-animal luminescent imaging was used to assess the effects of MH-I on the pulmonary uptake of the SARS-COV-2 pseudovirus.

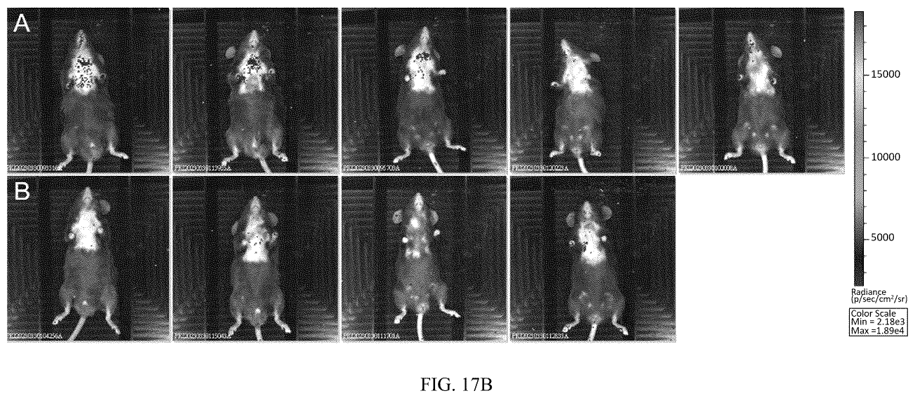

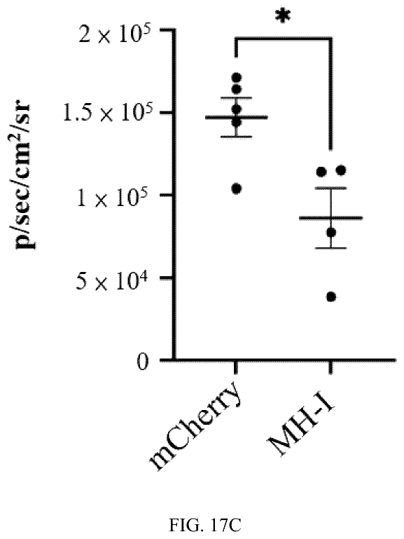

To determine the susceptibility of hACE2 mice to pseudoviruses expressing different variants of the SARS-COV-2 spike protein, various spike-variant pseudoviruses were intranasally injected into hACE2 mice. After 24 h, the luminescence signal was measured, and it was observed that the pseudovirus carrying the B.1.1.7 spike variant exhibited the highest sensitivity in the mice (FIG. 17A). Therefore, this specific variant was selected for further investigation in subsequent animal studies.

Compared to the mCherry-pretreated pseudovirus, which is used as control group and shown as the mice in the upper row in FIG. 17B, significant reduction (41.5%) in the pulmonary uptake of pseudovirus was observed in mice inoculated with the MH-I-pretreated pseudovirus (p<0.05, unpaired t test), shown as the mice in the lower row in FIG. 17B. This suggests that MH-I exerts a neutralizing effect against the pseudovirus in vivo. FIG. 17C shows the quantitative comparison of the pseudovirus pulmonary uptake between mice groups receiving mCherry or MH-I-pretreated pseudovirus (*p<0.05, stats method).

### Example 10. MH-I is a Safe Therapy

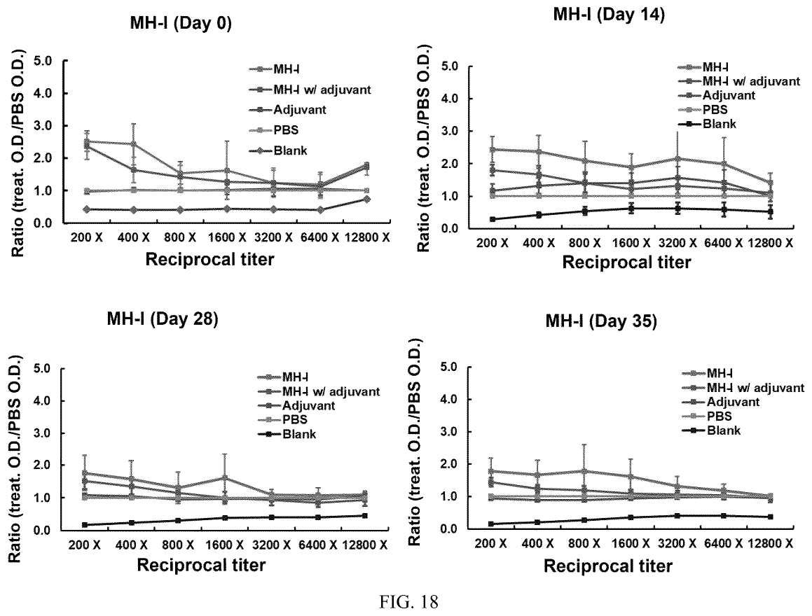

To evaluate safety of MH-I as a molecular therapy, subcutaneous immunization regimens were administered to mice with MH-I alone, MH-I in complete Freund's adjuvant, adjuvant only, and PBS on days 14, 28, and 35. The mice were monitored for 35 days, and 0.2 mL of blood was collected on days 0, 14, 28, and 35 post intraperitoneal injection. Serum was isolated and subjected to MH-I antibody detection via enzyme-linked immunosorbent assay (ELISA). The results were shown in FIG. 18, and the tests unequivocally indicated that neither MH-I alone nor MH-I in complete Freund's adjuvant elicited an anti-MH-I antibody response. These findings underscore the promising role of MH-I in molecular therapy and its potential to contribute to ongoing efforts to combat viral infections.

It will be understood that the above descriptions of embodiments are given by way of example only and that various modifications may be made by those having ordinary skill in the art. Although various embodiments of the disclosure have been described above with a certain degree of particularity, or with reference to one or more individual embodiments, those having ordinary skill in the art could make numerous alterations to the disclosed embodiments without departing from the scope of this disclosure.

What is claimed is:

1. A method for inhibiting combination between SARS-COV-2 and a cell of a subject in need thereof, comprising contacting the cell with an effective amount of a non-natural polypeptide including a fragment of major histocompatibility complex class I (MHC I).

2. The method of claim 1, wherein the non-natural polypeptide has at least 75% sequence identity to MHC I.

3. The method of claim 1, wherein the non-natural polypeptide has an amino acid sequence of SEQ ID NOs: 1, 2, 3, 4, 5, or 6.

4. The method of claim 1, wherein the non-natural polypeptide shows binding activity to a spike protein receptor binding domain of the SARS-COV-2.

5. The method of claim 1, wherein the non-natural polypeptide consists of 10 to 400 amino acids.

6. The method of claim 1, wherein the non-natural polypeptide inhibits the combination between the SARS-COV-2 and a cell membrane receptor of the cell.

7. The method of claim 6, wherein the cell membrane receptor of the cell is angiotensin-converting enzyme II (ACE2).

8. The method of claim 1, wherein the SARS-COV-2 is a wild-type or a variant thereof.

9. The method of claim 8, wherein the variant is any one selected from the group consisting of Alpha, Beta, Gamma, Delta, and Omicron.

10. A method for preventing or treating SARS-COV-2 infection in a subject in need thereof, comprising administering to the subject a pharmaceutical composition, wherein the pharmaceutical composition comprises an effective amount of a non-natural polypeptide including a fragment of major histocompatibility complex class I (MHC I) and a pharmaceutically acceptable carrier thereof.

11. The method of claim 10, wherein the non-natural polypeptide has at least 75% sequence identity to MHC I.

12. The method of claim 10, wherein the non-natural polypeptide has an amino acid sequence of SEQ ID NOs: 1, 2, 3, 4, 5, or 6.

13. The method of claim 10, wherein the non-natural polypeptide shows binding activity to a spike protein receptor binding domain of the SARS-COV-2.

14. The method of claim 10, wherein the non-natural polypeptide consists of 10 to 400 amino acids.

15. The method of claim 10, wherein the non-natural polypeptide inhibits combination between the SARS-COV-2 and a cell membrane receptor of a cell in the subject.

16. The method of claim 15, wherein the cell membrane receptor is angiotensin-converting enzyme II (ACE2).

17. The method of claim 10, wherein the SARS-COV-2 is a wild-type or a variant thereof.

18. The method of claim 17, wherein the variant is any one selected from the group consisting of Alpha, Beta, Gamma, Delta, and Omicron.

19. A non-natural polypeptide having at least 70% sequence identity to at least one of SEQ ID NOs: 1, 2, 3, 4, 5, or 6, and showing binding activity to a spike protein receptor binding domain of SARS-COV-2.

20. The non-natural polypeptide of claim 19, having an amino acid sequence of at least one of SEQ ID NOs: 1, 2, 3, 4, 5, or 6.
